(19) United States
(12) Patent Application Publication
(10) Pub. No.: US 2012/0270277 A1
(43) Pub. Date: Oct. 25, 2012

(54) Biomass Hydrolysis Process

(75) Inventors: Wang Han, Beijing (CN); Dongmin Li, Beijing (CN); Mengmeng Liu, Beijing (CN)

(73) Assignees: COFTCO CORPORATION, Beijing (CN); NOVOZYMES A/S, Bagsvaerd (DK)

(21) Appl. No.: 13/516,369

(22) PCT Filed: Dec. 21, 2010
(86) PCT No.: PCT/EP10/70337

§ 371 Date: Jul. 12, 2012

(30) Foreign Application Priority Data
Dec. 21, 2009 (CN) PCT/CN2009/075764

(51) Int. Cl. C12P 19/14; C12P 7/14

(52) U.S. Cl. 435/99; 435/162

(57) ABSTRACT

The invention relates to a biomass process comprising split dosage of the cellulose hydrolysing enzyme.

### TECHNICAL FIELD

The present invention relates to processes comprising use of split dosing of cellulose hydrolyzing enzyme in hydrolysis of lignocellulose-containing materials.

### BACKGROUND OF THE INVENTION

Due to the limited reserves of fossil fuels and worries about emission of greenhouse gasses there is an increasing focus on using renewable energy sources, e.g. fermentation products, such as bioethanol. Production of ethanol from biomass, i.e. lignocellulose-containing material, is known in the art and may comprise pretreatment, enzymatic hydrolysis, and fermentation of the lignocellulose-containing material into ethanol. The cost of enzymes used in the hydrolysis has been regarded as limiting for the profitability of such processes. Consequently, there is a need for providing improved and more efficient processes for enzymatic hydrolysis of lignocellulose-containing material into substrates suitable for fermentation.

The inventors of the present application have now surprisingly found that by splitting the cellulose hydrolyzing enzymes in at least two dosages and adding the dosages at different stages the hydrolysis of high dry solids biomass slurries can be significantly improved.

### SUMMARY OF THE INVENTION

The present invention relates to a process comprising enzymatic hydrolysis of a lignocellulose-containing material at a high dry solids concentration, and optionally fermentation of the hydrolysate into a fermentation product, preferably ethanol, wherein the cellulose hydrolyzing enzymes are added at two or more stages in the process.

Accordingly, in a first aspect the invention relates to a process for producing a hydrolyzate from lignocellulose-containing material, comprising the steps of, (a) pre-treating lignocellulose-containing material; (b) forming a slurry comprising water, pre-treated lignocellulose-containing material and cellulose hydrolysing enzymes, (c) and incubating the slurry, (d) adding more hydrolyzing enzymes, and (e) incubating the slurry, to produce a hydrolysate, wherein the slurry has a dry solids concentration of at least 25%.

### DETAILED DESCRIPTION OF THE INVENTION

### Lignocellulose-Containing Material

The term “lignocellulose-containing materials” used herein refer to a material primarily consisting of cellulose, hemicellulose, and lignin. Lignocellulose-containing materials are often referred to as “biomass”.

The structure of lignocellulose is not directly accessible to enzymatic hydrolysis. Therefore, the lignocellulose has to be pretreated, e.g. by acid hydrolysis under adequate conditions of pressure, humidity and temperature, in order to break the lignin seal and disrupt the crystalline structure of cellulose. This causes solubilization and saccharification of the hemicellulose fraction. The cellulose fraction can then be hydrolyzed, e.g. enzymatically by cellulase enzymes, to convert the carbohydrate polymers into mono- and oligosaccharides, which may be fermented into a desired fermentation product, such as ethanol. Optionally the fermentation product is recovered, e.g. by distillation.

Any lignocellulose-containing material is contemplated according to the present invention. The lignocellulose-containing material may be any material containing lignocellulose. In a preferred embodiment the lignocellulose-containing material contains at least 30 wt-%, preferably at least 50 wt.-%, more preferably at least 70 wt-%, even more preferably at least 90 wt-% lignocellulose. It is to be understood that the lignocellulose-containing material may also comprise other constituents such as cellulosic material, including cellulose and hemicellulose, and may also comprise other constituents such as proteinaceous material, starch, sugars, such as fermentable sugars and/or un-fermentable sugars.

Lignocellulose-containing material is generally found, for example, in the stems, leaves, hulls, husks, and cobs of plants or leaves, branches, and wood of trees. Lignocellulose-containing material can be, but is not limited to, herbaceous material, agricultural residues, forestry residues, municipal solid wastes, waste paper, and pulp and paper mill residues. It is understood herein that lignocellulose-containing material may be in the form of plant cell wall material containing lignin, cellulose, and hemicellulose in a mixed matrix.

The lignocellulose-containing material may comprise corn stover, hard wood, such as poplar and birch, soft wood such as pine wood, switch grass, cereal straw and/or husks, such as straw from rice, wheat, barley rye etc., municipal solid waste (MSW), industrial organic waste, office paper, wood chips, bagasse, paper or pulp processing waste or mixtures thereof.

In a preferred embodiment the lignocellulose-containing material is corn stover. In another preferred aspect, the lignocellulose-containing material is corn fibre.

### Pretreatment

The lignocellulose-containing material may be pretreated in any suitable way. Pretreatment is carried out before hydrolysis and/or fermentation. The goal of pretreatment is to separate and/or release cellulose, hemicellulose and/or lignin and this way improve the rate of hydrolysis. Pretreatment methods such as wet-oxidation and alkaline pretreatment targets lignin, while dilute acid and auto-hydrolysis targets hemicellulose. Steam explosion is an example of a pretreatment that targets cellulose.

According to the invention pretreatment step (a) may be a conventional pretreatment step using techniques well known in the art. In a preferred embodiment pretreatment takes place in an aqueous slurry. The lignocellulose-containing material may during pretreatment be present in an amount between 10-80 wt.-%, preferably between 20-70 wt-%, especially between 30-60 wt.-%, such as around 50 wt-%.

The pretreatment is carried out prior to the hydrolysis and/or fermentation.

The term “chemical treatment” refers to any chemical pretreatment which promotes the separation and/or release of cellulose, hemicellulose and/or lignin. Examples of suitable chemical pretreatments include treatment with; for example, dilute acid, lime, alkaline, organic solvent, ammonia, sulphur dioxide, carbon dioxide. Further, wet oxidation and pH-controlled hydrothermolysis are also considered chemical pretreatment.

In a preferred embodiment the chemical pretreatment is acid treatment, more preferably, a continuous dilute and/or mild acid treatment, such as, treatment with sulphuric acid, or another organic and/or inorganic acid, such as acetic acid, citric acid, tartaric acid, succinic acid, hydrogen chloride or mixtures thereof. Other acids may also be used. Mild acid treatment means that the treatment pH lies in the range from 1-5, preferably pH 1-3. In a specific embodiment the acid concentration is in the range from 0.1 to 2.0 wt % acid, preferably sulphuric acid. The acid may be contacted with the lignocellulose-containing material and the mixture may be held at a temperature in the range of 160-220° C., such as 165-195° C., for periods ranging, e.g. 1-60 minutes, such as 2-30 minutes or 3-12 minutes. Addition of strong acids, such as sulphuric acid, may be applied to remove hemicellulose. This enhances the digestibility of cellulose.

Other techniques are also contemplated. Cellulose solvent treatment has been shown to convert about 90% of cellulose to glucose. It has also been shown that enzymatic hydrolysis could be greatly enhanced when the lignocellulose structure is disrupted. Alkaline H₂O₂, ozone, organosolv (uses Lewis acids, FeCl₃, (Al)₂SO₄ in aqueous alcohols), glycerol, dioxane, phenol, or ethylene glycol are among solvents known to disrupt cellulose structure and promote hydrolysis (Mosier et al. Bioresource Technology 96 (2005), p. 673-686).

Alkaline chemical pretreatment with base, e.g. NaOH, Na₂CO₃ and/or ammonia or the like, is also contemplated according to the invention. Pretreatments method using ammonia is described in, e.g. WO2006110891, WO200611899, WO200611900, WO2006110901 (which are hereby incorporated by reference).

Wet oxidation techniques involve use of oxidizing agents, such as: sulphite based oxidizing agents or the like. Examples of solvent pretreatments include treatment with DMSO (Dimethyl Sulphoxide) or the like. Chemical pretreatment is generally carried out for 1 to 60 minutes, such as from 5 to 30 minutes, but may be carried out for shorter or longer periods of time dependent on the material to be pretreated.

Other examples of suitable pretreatment methods are described by Schell et al. (2003) Appl. Biochem and Biotechn. Vol. 105-108, p. 69-85, Mosier et al. Bioresource Technology 96 (2005) 673-686, Ahring et al. in WO2006032282 and WO200160752, Foody et al. in WO2006034590, and Ballesteros et al. in US publication no. 20020164730, which references are hereby all incorporated by reference.

The term “mechanical pretreatment” refers to any mechanical (or physical) treatment which promotes the separation and/or release of cellulose, hemicellulose and/or lignin from lignocellulose-containing material. For example, mechanical pretreatment includes various types of milling, irradiation, steaming/steam explosion, wet oxidation, and other hydrothermal treatments.

Mechanical pretreatment includes comminution (mechanical reduction of the size). Comminution includes dry milling, wet milling and vibratory ball milling. Mechanical pretreatment may involve high pressure and/or high temperature (steam explosion). In an embodiment of the invention high pressure means pressure in the range from 300 to 600 psi, preferably 400 to 500 psi, such as around 450 psi. In an embodiment of the invention high temperature means temperatures in the range from about 100 to 300° C., preferably from about 140 to 235° C. In a preferred embodiment mechanical pretreatment is a batch-process, steam gun hydrolyzer system which uses high pressure and high temperature as defined above. A Sunds Hydrolyzer (available from Sunds Defibrator AB (Sweden) may be used for this.

In a preferred embodiment both chemical and mechanical pretreatments are carried out. For instance, the pretreatment step may involve dilute or mild acid treatment and high temperature and/or pressure treatment. The chemical and mechanical pretreatment may be carried out sequentially or simultaneously, as desired.

Accordingly, in a preferred embodiment, the lignocellulose-containing material is subjected to both chemical and mechanical pretreatment to promote the separation and/or release of cellulose, hemicellulose and/or lignin.

In a preferred embodiment a mechanical pretreatment is carried out before a stream explosion pretreatment.

In a preferred embodiment the pretreatment is carried out as a dilute and/or mild acid steam explosion step. In another preferred embodiment pretreatment is carried out as an ammonia fiber explosion step (or AFEX pretreatment step).

As used in the present invention the term “biological pretreatment” refers to any biological pretreatment which promotes the separation and/or release of cellulose, hemicellulose, and/or lignin from the lignocellulose-containing material. Biological pretreatment techniques can involve applying lignin-solubilizing microorganisms (see, for example, Hsu, T.-A., 1996, Pretreatment of biomass, in Handbook on Bioethanol: Production and Utilization, Wyman, C. E., ed., Taylor & Francis, Washington, D.C., 179-212; Ghosh, P., and Singh, A., 1993, Physicochemical and biological treatments for enzymatic/microbial conversion of lignocellulosic biomass, Adv. Appl. Microbiol. 39: 295-333; McMillan, J. D., 1994, Pretreating lignocellulosic biomass: a review, in Enzymatic Conversion of Biomass for Fuels Production, Himmel, M. E., Baker, J. O., and Overend, R. P., eds., ACS Symposium Series 566, American Chemical Society, Washington, D.C., chapter 15; Gong, C. S., Cao, N. J., Du, J., and Tsao, G. T., 1999, Ethanol production from renewable resources, in Advances in Biochemical Engineering/Biotechnology, Scheper, T., ed., Springer-Verlag Berlin Heidelberg, Germany, 65: 207-241; Olsson, L., and Hahn-Hagerdal, B., 1996, Fermentation of lignocellulosic hydrolysates for ethanol production, Enz. Microb. Tech. 18: 312-331; and Vallander, L., and Eriksson, K.-E. L., 1990, Production of ethanol from lignocellulosic materials: State of the art, Adv. Biochem. Eng./Biotechnol. 42: 63-95).

### Washing of Pretreated Lignocellulose-Containing Material

When lignocellulose-containing material is pretreated, degradation products that are inhibitory to enzymes may be produced. Washing of pretreated lignocellulose-containing material in order to remove inhibitors of enzymes may improve the enzymatic hydrolysis.

The inhibitors are lignocellulose degradation products including lignin degradation products, cellulose degradation products and hemicellulose degradation products. The lignin degradation products may be phenolic in nature. The hemicellulose degradation products include furans from sugars (such as hexoses and/or pentoses), including mannose, galactose, rhamanose, arabinose and xylose, including oligosaccharides. The compounds inhibitory to enzymes are believed to include xylooligosaccharides (XOOs) or complexes of XOO and soluble lignin, present in the PCS liquor. According to the present invention soluble compounds inhibitory to enzymes are removed from the pretreated lignocellulose-containing material by washing with a washing solution. The washing solution is preferably an aqueous washing solution. The washing solution may be a substantially pure solution of water, or water with a significant amount of additives, e.g. such as a detergent and/or an organic solvent to improve the extraction and/or solubility of the compounds inhibitory to enzymes.

### Hydrolysis

Before and/or simultaneously with fermentation the pretreated and washed lignocellulose-containing material is enzymatically hydrolyzed to break down cellulose and hemicellulose into sugars and/or oligosaccharides.

Hydrolysis may in a preferred embodiment be carried out as a fed batch process where the pretreated lignocellulose-containing material (substrate) is fed gradually to an, e.g., enzyme containing hydrolysis solution. The pretreated lignocellulose-containing material may be supplied to the enzyme containing hydrolysis solution either in one or more distinct batches, as one or more distinct continuous flows or as a combination of one or more distinct batches and one or more distinct continuous flows. The dry solids concentration throughout the hydrolysis is at least 20%, more preferably at least 25%, at least 26%, at least 27%, at least 28%, at least 29% or even at least 30%.

According to the invention the pretreated lignocellulose-containing material is hydrolyzed using at least the enzymes endo-glucanases (EC 3.2.1.4); cellobiohydrolases (EC 3.2.1.91), and beta-glucosidases (EC 3.2.1.21).

Additional enzymes which may be applied during hydrolysis are described in the “Enzymes”-section below, and include xylanase, arabinofuranosidase, acetyl xylan esterase, ferulic acid esterase, glucuronidases, endo-galactanase, mannase, endo- or exo-arabinases, endo- or exo-galactanases, and mixtures of two or more thereof.

The enzyme(s) used for hydrolysis is(are) capable of directly or indirectly converting the washed pretreated lignocellulose-containing material into fermentable sugars which can be fermented into a desired fermentation product, such as ethanol.

According to the invention the amount of enzymes is split in at least two dosages of which the first is applied at the initiation of the hydrolysis step, and the remaining dosage(s) is(are) applied later during the hydrolysis.

In a preferred embodiment the amount of enzymes is split in two dosages of approximately equal size, the first half is applied at the initiation of the hydrolysis step, and the slurry is incubated for around 12-48 hrs, preferably for around 18-36 hrs, more preferably for around 20-30 hrs before the remaining dosage is applied. The hydrolysis is continued for an additional 48-72 hrs.

Embodiments wherein the amount of enzymes is split in several dosages of approximately equal and/or different size and applied at approximately regular and/or irregular intervals during the hydrolysis are likewise contemplated. The enzymes may even be applied as a continuous addition of a dilute enzyme preparation during the hydrolysis or at least the initial half of the hydrolysis.

The full duration of the hydrolysis, i.e., the process steps (c)+(e), is preferably between 72-120 hrs.

Without being bound by theory, it is believed that the positive effect from using split enzyme dosage is because the enzymes, especially the cellobiohydrolase I and cellobiohydrolase II and to a lesser degree also the endo-glucanases, in general are inactivated during the hydrolysis step. That is why the positive effect is observed at high dry solid concentrations where the shear forces acting on the enzymes are stronger. By using split enzyme dosing and delaying adding one or more dosage(s) into the incubation the remaining enzyme activity in the later stages of the hydrolysis is increased.

Enzymatic treatment is carried out in a suitable aqueous environment under conditions which can readily be determined by one skilled in the art. Preferably, hydrolysis is carried out at a temperature between 25° C. and 70° C., preferably between 40° C. and 60° C., especially around 50° C. The process is preferably carried out at a pH in the range from 3-8, preferably pH 4-6, especially around pH 5.

The hydrolyzate may in one embodiment be fermented to produce a fermentation product. It is contemplated that hydrolysis and fermentation may be carried out simultaneously (SHHF process) or sequentially (SHF process).

### Fermentation

According to the invention the pretreated (and hydrolyzed) lignocellulose-containing material is fermented by at least one fermenting organism capable of fermenting fermentable sugars, such as glucose, xylose, mannose, and galactose directly or indirectly into a desired fermentation product.

The fermentation is preferably ongoing for between 8 to 96 hours, preferably 12 to 72, more preferable from 24 to 48 hours. In an embodiment the fermentation is carried out at a temperature between 20 to 40° C., preferably 26 to 34° C., in particular around 32° C. In an embodiment the pH is from pH 3 to 6, preferably around pH 4 to 5.

Preferred for ethanol fermentation is yeast of the species Saccharomyces cerevisiae, preferably strains which are resistant towards high levels of ethanol, i.e., up to, e.g., about 10, 12 or 15 vol. % ethanol or more, such as 20 vol. % ethanol.

Contemplated according to the invention is simultaneous hydrolysis and fermentation (SHF). In an embodiment there is no separate holding stage for the hydrolysis, meaning that the hydrolysing enzyme(s) and the fermenting organism are added together. When the fermentation is performed simultaneous with hydrolysis the temperature is preferably between 26° C. and 35° C., more preferably between 30° C. and 34° C., such as around 32° C. A temperature program comprising at least two holding stages at different temperatures may be applied according to the invention.

During washing of the pretreated lignocellulose-containing material dissolved sugars may accumulate in a recycled aqueous washing solution. The dissolved sugars will comprise C5 sugars from the degradation of the hemicellulose, such as xylose. These sugars can be fermented with a suitable fermenting organism which is able to convert C5 sugars into a desired fermentation product. This C5 fermentation may be performed separately, or the dissolved sugars accumulated in the recycled aqueous washing solution may be added to the hydrolyzed lignocellulose-containing material for a combined C6 and C5 fermentation. Such a fermentation is preferably performed with at least one organism able to ferment both C6 (e.g., glucose) and C5 sugars (e.g., xylose). Alternatively, fermentation may be performed with at least two separate organisms, each optimized to utilizing either the C6 or the C5 sugars, either in one fermentation step under conditions allowing both organisms to ferment, or as at least two fermentation steps, each under conditions allowing one of the organisms to ferment,

The process of the invention may be performed as a batch, fed-batch or as a continuous process. Preferably the fermentation step is performed as a continuous fermentation.

### Recovery

Subsequent to fermentation the fermentation product may be separated from the fermentation broth. The broth may be distilled to extract the fermentation product or the fermentation product may be extracted from the fermentation broth by micro or membrane filtration techniques. Alternatively the fermentation product may be recovered by stripping. Recovery methods are well known in the art.

### Fermentation Products

The process of the invention may be used for producing any fermentation product. Especially contemplated fermentation products include alcohols (e.g., ethanol, methanol, butanol); organic acids (e.g., citric acid, acetic acid, itaconic acid, lactic acid, gluconic acid); ketones (e.g., acetone); amino acids (e.g., glutamic acid); gases (e.g., H₂ and CO₂); antibiotics (e.g., penicillin and tetracycline); enzymes; vitamins (e.g., riboflavin, B12, beta-carotene); and hormones.

Also contemplated products include consumable alcohol industry products, e.g., beer and wine. In a preferred embodiment the fermentation product is an alcohol, especially ethanol. The fermentation product, such as ethanol, obtained according to the invention, may preferably be fuel alcohol/ethanol. However, in the case of ethanol it may also be used as potable ethanol.

### Fermenting Organism

The term “fermenting organism” refers to any organism, including bacterial and fungal organisms, suitable for producing a desired fermentation product. Especially suitable fermenting organisms according to the invention are able to ferment, i.e., convert, sugars, such as glucose, directly or indirectly into the desired fermentation product. Also suitable are fermenting organisms capable of converting C5 sugars such as xylose into a desired fermentation product. Examples of fermenting organisms include fungal organisms, especially yeast. Preferred yeast includes strains of Saccharomyces spp., in particular a strain of Saccharomyces cerevisiae or Saccharomyces uvarum; a strain of Pichia, preferably Pichia stipitis, such as Pichia stipitis CBS 5773; a strain of Candida, in particular a strain of Candida utilis, Candida diddensii, or Candida boidinii. Other contemplated yeast includes strains of Zymomonas; Hansenula, in particular H. anomala; Klyveromyces, in particular K. fragilis; and Schizosaccharomyces, in particular S. pombe.

Commercially available yeast includes, e.g., ETHANOL RED™ yeast (available from Fermentis/Lesaffre, USA), FALI (available from Fleischmann's Yeast, USA), SUPERSTART and THERMOSACC™ fresh yeast (available from Ethanol Technology, WI, USA), BIOFERM AFT and XR (available from NABC—North American Bioproducts Corporation, GA, USA), GERT STRAND (available from Gert Strand AB, Sweden), and FERMIOL (available from DSM Specialties). ANQI YEAST (available from Anqi yeast (CHIFENG) CO., LTD, China).

### Enzymes

Even though not specifically mentioned in the context of a process of the invention, it is to be understood that the enzymes (as well as other compounds) are used in an “effective amount”.

Cellulases: The term “cellulases” as used herein are understood as comprising the cellobiohydrolases (EC 3.2.1.91), e.g., cellobiohydrolase I and cellobiohydrolase II, as well as the endo-glucanases (EC 3.2.1.4) and beta-glucosidases (EC 3.2.1.21). Cellulases are applied in the hydrolysis step.

In order to be efficient, the digestion of cellulose and hemicellulose requires several types of enzymes acting cooperatively. At least three categories of enzymes are necessary to convert cellulose into fermentable sugars: endo-glucanases (EC 3.2.1.4) that cut the cellulose chains at random; cellobiohydrolases (EC 3.2.1.91) which cleave cellobiosyl units from the cellulose chain ends and beta-glucosidases (EC 3.2.1.21) that convert cellobiose and soluble cellodextrins into glucose. Among these three categories of enzymes involved in the biodegradation of cellulose, cellobiohydrolases are the key enzymes for the degradation of native crystalline cellulose. The term “cellobiohydrolase I” is defined herein as a cellulose 1,4-beta-cellobiosidase (also referred to as Exo-glucanase, Exo-cellobiohydrolase or 1,4-beta-cellobiohydrolase) activity, as defined in the enzyme class EC 3.2.1.91, which catalyzes the hydrolysis of 1,4-beta-D-glucosidic linkages in cellulose and cellotetraose, by the release of cellobiose from the non-reducing ends of the chains. The definition of the term “cellobiohydrolase II activity” is identical, except that cellobiohydrolase II attacks from the reducing ends of the chains.

Endoglucanases (EC No. 3.2.1.4) catalyses endo hydrolysis of 1,4-beta-D-glycosidic linkages in cellulose, cellulose derivatives (such as carboxy methyl cellulose and hydroxy ethyl cellulose), lichenin, beta-1,4 bonds in mixed beta-1,3 glucans such as cereal beta-D-glucans or xyloglucans and other plant material containing cellulosic parts. The authorized name is endo-1,4-beta-D-glucan 4-glucano hydrolase, but the abbreviated term endoglucanase is used in the present specification.

The cellulase activity may, in a preferred embodiment, be derived from a fungal source, such as a strain of the genus Trichoderma, preferably a strain of Trichoderma reesei; a strain of the genus Humicola, such as a strain of Humicola insolens; or a strain of Chrysosporium, preferably a strain of Chrysosporium lucknowense.

In a preferred embodiment the cellulase preparation comprising a polypeptide having cellulolytic enhancing activity (GH61A), preferably the one disclosed in WO2005074656. The cellulase preparation may further comprise a beta-glucosidase, such as the fusion protein disclosed in U.S. 60/832,511. In an embodiment the cellulase preparation also comprises a CBH II, preferably Thielavia terrestris cellobiohydrolase II CEL6A. In an embodiment the cellulase preparation also comprises a cellulase enzymes derived from Trichoderma reesei. In a preferred embodiment the cellulase preparation is the cellulase preparation used in Example 1 and disclosed in WO 2008/151079, which cellulase preparation comprises cellulolytic enzymes derived from Trichoderma reesei, a polypeptide having cellulolytic enhancing activity (GH61A) disclosed in WO2005074656, and an Aspergillus fumigatus beta-glucosidase disclosed in WO 2008/151079.

The cellulase may be a commercially available product, e.g. CELLUCLAST®, 1.5 L or CELLUZYME™, or Cellic Ctec™ (all from Novozymes NS, Denmark) or ACCELLERASE™ 1000 or ACCELLERASE™ 1500 (all from Genencor Int.).

The cellulase may be dosed in the range from 0.1-100 FPU per gram dry solids (DS), preferably 0.5-50 FPU per gram DS, especially 1-20 FPU per gram DS. The cellulase may be dosed in the range from 0.1-10000 mg enzyme protein (EP)/kg dry solids (DS), preferably 0.5-5000 mg EP/kg DS, especially 1-2500 mg EP/kg DS.

Hemicellulases: Hemicellulose can be broken down by hemicellulases and/or acid hydrolysis to release its five and six carbon sugar components.

Any hemicellulase suitable for use in hydrolyzing hemicellulose may be used. Preferred hemicellulases include xylanase, arabinofuranosidase, acetyl xylan esterase, ferulic acid esterase, glucuronidases, endo-galactanase, mannase, endo or exo arabinases, endo or exo galactanases, and mixtures of two or more thereof. Preferably, the hemicellulase for use in the present invention is an exo-acting hemicellulase, and more preferably, the hemicellulase is an exo-acting hemicellulase which has the ability to hydrolyze hemicellulose under acidic conditions of below pH 7, preferably pH 3-7. Examples of hemicellulase compositions suitable for use in the present invention include VISCOZYME™, and ULTRAFLO™ (available from Novozymes A/S, Denmark).

Xylanase (EC 3.2.1.8) for use in the present invention is preferably an endo-1,4-beta-xylanase, and preferably of Glycoside Hydrolase Family 10 or 11 (GH10 or GH11). The GH10 or GH11 are defined in Cantarel et al. (2008) in Nucl. Acids Res. 2009 37: D233-D238 and on www.cazy.org.

The xylanase may be of any origin including mammalian, plant or animal origin; however, it is preferred that the xylanase is of microbial origin. In particular the xylanase may be one derivable from a filamentous fungus or a yeast. Preferably the xylanase is derived from a filamentous fungus such as from Aspergillus sp., Bacillus sp., Humicola sp., Myceliophotora sp., Poitrasia sp. Rhizomucorsp. or Trichoderma. The xylanase is preferably a GH10 xylanase. Most preferred is a xylanase derived from Aspergillus aculeatus and disclosed as xylanase II in WO 1994/021785.

The xylanase applied in the process of the present invention for treating the used washing solution is preferably an immobilized xylanase. If a non-immobilized xylanase is used it is added in amounts of 0.001-1.0 g/kg DS substrate, preferably in the amounts of 0.005-0.5 g/kg DS substrate, and most preferably from 0.05-0.10 g/kg DS substrate.

A xylanase may also be added in the hydrolysis step of the present invention in amounts of 0.001-1.0 g/kg DS substrate, preferably in the amounts of 0.005-0.5 g/kg DS substrate, and most preferably from 0.05-0.10 g/kg DS substrate.

Ferulic acid esterase (EC 3.1.1.73) catalyses the hydrolysis of the 4-hydroxy-3-methoxycinnamoyl (feruloyl) group from an esterified sugar, which is usually arabinose in arabinoxylan. A suitable ferulic acid esterase may be obtained from a strain of a filamentous fungus (e.g., Trichoderma, Meripilus, Humicola, Aspergillus, Fusarium) or from a bacteria (e.g. Bacillus), such as from Aspergillus niger, e.g., the FAEIII ferulic acid esterase described by Faulds et al. 1994, Microbiology, 140, pp. 779-787.

Arabinofuranosidase (EC 3.2.1.55) catalyses the hydrolysis of terminal non-reducing alpha-L-arabinofuranoside residues in alpha-L-arabinosides.

Galactanase (EC 3.2.1.89), arabinogalactan endo-1,4-beta-galactosidase, catalyses the endohydrolysis of 1,4-D-galactosidic linkages in arabinogalactans.

Pectinase (EC 3.2.1.15) catalyses the hydrolysis of 1,4-alpha-D-galactosiduronic linkages in pectate and other galacturonans.

Xyloqlucanase catalyses the hydrolysis of xyloglucan.

The hemicellulase may be added in an amount effective to hydrolyze hemicellulose, such as, in amounts from about 0.001 to 0.5 wt.-% of dry solids (DS), more preferably from about 0.05 to 0.5 wt.-% of DS.

### Materials & Methods

### Enzymes:

A cellulase preparation comprising cellulolytic enzymes derived from Trichoderma reesei, a polypeptide having cellulolytic enhancing activity (GH61A) disclosed in WO2005074656, and an Aspergillus fumigatus beta-glucosidase disclosed in WO 2008/151079. The cellulase preparation is disclosed in WO 2008/151079.

### Example 1

Corn stover was pretreated using steam explosion at 205° C. for 5 minutes. The resulting PCS (Pretreated Corn Stover) was washed using tap water to remove soluble inhibitors. A fed-batch hydrolysis was performed with washed PCS at 15%, 20% and 30% TS (Total Solids), respectively. All fed-batch of washed PCS were started at 12.6% TS and 1, 3 or 4 additional loadings of PCS were required to reach designated final TS of 15%, 20% or 30%. Cellulase preparation was added at an enzyme dosage of 6 mg EP/g cellulose at 0 hour or split into two equal halves and half was added at 0 hour and the other half at 24 hours. The hydrolysis was performed at 50° C. and pH 5.0 for 96 hours.

Samples of whole slurry were drawn every 24 hours and sugar concentrations were determined by HPLC. The results are shown in table 1. The glucose equivalent conversion is calculated as the percentage of produced cellobiose and glucose out of the total glucose potential in PCS.

TABLE 1
Comparasion of sugar concentration and % glucose equivalent conversion
following hydrolysis with one dosage or split dosage of enzymes

| | | | | | | 96 hours | | |
|---|---|---|---|---|---|---|---|---|
| | | | | | | | | Glucose |
| | | 48 hours | | 72 hours | | | | Equivalent |
| | | cellobiose | | cellobiose | glucose | cellobiose | glucose | Conversion |
| | | (g/L) | glucose (g/L) | (g/L) | (g/L) | (g/L) | (g/L) | (%) |
| 15% | one | 6.41 | 52.78 | 7.63 | 58.66 | 6.91 | 67.69 | 78.2 |
| TS | dosage | | | | | | | |
| | split | 3.72 | 44.36 | 5.43 | 53.28 | 6.46 | 58.38 | 68.0 |
| | dosage | | | | | | | |
| 20% | one | 9.33 | 62.22 | 11.95 | 69.92 | 12.85 | 75.29 | 69.5 |
| TS | dosage | | | | | | | |
| | split | 7.34 | 54.62 | 8.35 | 62.04 | 9.39 | 67.58 | 60.6 |
| | dosage | | | | | | | |
| 30% | one | 9.38 | 79.09 | 18.26 | 93.65 | 23.21 | 99.85 | 64.8 |
| TS | dosage | | | | | | | |
| | split | 9.34 | 71.12 | 18.78 | 87.82 | 27.37 | 101.86 | 68.2 |
| | dosage | | | | | | | |

### Example 2

The test described above was repeated in order to obtain data around a TS content of 25% except that the reactor setup was slightly different. In Example 1 a vertical reactor setup was used, whereas in the present example a horizontal reactor was used. In general this will result in a lower sugar yield.

TABLE 2
Comparasion of sugar concentration and % glucose equivalent conversion
following hydrolysis with one dosage or split dosage of enzymes

| | | | | | | | | Glucose |
|---|---|---|---|---|---|---|---|---|
| | | 48 hours | | 72 hours | | 96 hours | | Equivalent |
| | | cellobiose | glucose | cellobiose | glucose | cellobiose | glucose | conversion % |
| 25% | one dosage | 6.61 | 50.52 | 7.91 | 58.53 | 8.52 | 62.53 | 40.54 |
| TS | split dosage | 5.16 | 45.97 | 6.33 | 53.59 | 13.10 | 64.73 | 46.34 |
| 28% | one dosage | 12.12 | 67.87 | 12.00 | 69.08 | 10.32 | 72.74 | 45.81 |
| TS | split dosage | 14.29 | 60.71 | 15.57 | 68.64 | 15.45 | 76.02 | 52.03 |

1-13. (canceled)

14. A process for producing a hydrolyzate, comprising:
(a) pre-treating a lignocellulose-containing material;
(b) forming a slurry comprising water, pre-treated lignocellulose-containing material and cellulose hydrolyzing enzymes,
(c) incubating the slurry,
(d) adding more hydrolyzing enzymes, and
(e) incubating the slurry, to produce a hydrolysate,
wherein the slurry has a dry solids concentration of at least 25%.

15. The process of claim 14, wherein the slurry of step (c) is incubating for around 12-48 hours before step (d).

16. The process of claim 14, wherein the duration of steps (c) and (e) is around 72-120 hours.

17. The process of claim 14, wherein the cellulose hydrolyzing enzymes comprise one or more of cellobiohydrolase I, cellobiohydrolase II and endo-glucanase.

18. The process of claim 14, further comprising fermenting the hydrolyzate of step (e) to produce a fermentation product.

19. The process of claim 18, wherein the hydrolysis and fermentation are simultaneous.

20. The process of claim 18, further comprising recovery of the fermentation product.

21. The process of claim 18, wherein the fermentation product is ethanol.

22. The process of claim 14, wherein the lignocellulose-containing material originates from materials selected from the group comprising: corn stover, corn fiber, hard wood, such as poplar and birch, soft wood, cereal straw, such as, wheat straw, switch grass, rice hulls, municipal solid waste, industrial organic waste, office paper, and mixtures thereof.

23. The process of claim 14, wherein the pretreatment in step (a) is acid pretreatment carried out using an organic and/or inorganic acid.

24. The process of claim 14, wherein the lignocellulose-containing material in step (a) is acid pretreated with from 0.1 to 2.0 wt. % acids.

25. The process of claim 14, wherein the lignocellulose-containing material in step (a) is mechanically pretreated at a high temperature and/or at high pressure.

26. The process of claim 14, wherein the lignocellulose-containing material in step (a) is pretreated by steam explosion, dilute acid steam explosion and/or wet oxidation.
